(19) United States
(12) Patent Application Publication
(10) Pub. No.: US 2018/0289278 A1
(43) Pub. Date: Oct. 11, 2018

(54) CARDIAC CATHETER INSPECTION APPARATUS, CARDIAC CATHETER INSPECTION SYSTEM, AND CARDIAC CATHETER INSPECTION METHOD

(71) Applicants: NIHON KOHDEN CORPORATION, Shinjuku-ku, Tokyo (JP); NATIONAL CEREBRAL & CARDIOVASCULAR CENTER, Suita-shi, Osaka (JP)

(72) Inventors: Kazuo NAKAZAWA, Suita-shi, Osaka (JP); Shin INADA, Suita-shi, Osaka (JP); Tatsuo NISHIHARA, Shinjuku-ku, Tokyo (JP); Koji TAKIZAWA, Shinjuku-ku, Tokyo (JP); Hiroki YUZE, Shinjuku-ku, Tokyo (JP); Naoki KOURA, Shinjuku-ku, Tokyo (JP)

(73) Assignees: NIHON KOHDEN CORPORATION, Shinjuku-ku, Tokyo (JP); NATIONAL CEREBRAL & CARDIOVASCULAR CENTER, Suita-shi, Osaka (JP)

(21) Appl. No.: 15/542,156

(22) PCT Filed: Jan. 5, 2016
(86) PCT No.: PCT/JP2016/000029

§ 371 Date: Jul. 7, 2017

(30) Foreign Application Priority Data
Jan. 9, 2015 (JP) 2015-003225

(51) Int. Cl. A61B 5/0452; A61B 5/024; A61B 5/0245; A61B 5/04; A61B 5/0402; A61B 5/0428; A61B 18/14; A61B 5/0404

(52) U.S. Cl. CPC A61B 5/04525; A61B 5/02405; A61B 5/0245; A61B 5/04012; A61B 2018/00351; A61B 5/04286; A61B 18/1492; A61B 5/0404; A61B 5/04028

(57) ABSTRACT

A cardiac catheter inspection apparatus comprising: an acquiring section (31) which is configured to acquire a first waveform of heart beats during sinus rhythm, and an arrhythmia waveform during arrhythmia; a measuring section (31) which is configured to measure a second electrocardiogram; a first calculating section (32) which is configured to calculate correction coefficients for statistically matching the first electrocardiogram and the second electrocardiogram with each other; a waveform producing section (34) which is configured to produce a comparison waveform that functions as an object to be compared with a response waveform of pacing performed by a cardiac catheter; and a second calculating section (36) which is configured to calculate a matching rate of the response waveform and the comparison waveform.

### TECHNICAL FIELD

The present disclosure relates to a cardiac catheter inspection apparatus, cardiac catheter inspection system, and cardiac catheter inspection method which identify the position of a focus for arrhythmia.

### BACKGROUND ART

It is known that, when arrhythmia such as ventricular premature contraction occurs, the arrhythmia is treated by selectively performing ablation on the site of abnormal activation which causes the arrhythmia, by using a cardiac catheter. In order to perform the treatment, it is important to correctly identify the position of a focus causing arrhythmia. For example, Patent Literature 1 below discloses an apparatus having a function of identifying the position of a focus causing arrhythmia.

In the case where ablation treatment is to be performed by using the apparatus disclosed in Patent Literature 1, the doctor firstly attaches electrodes to the patient who is in a supine posture on a bed in a cardiac catheter inspection room, and measures an electrocardiogram of the patient. From the electrocardiogram which is obtained in the measurement, a signal waveform of arrhythmia spontaneously occurring in the heart of the patient is acquired. During the catheter treatment, the acquired signal waveform of arrhythmia is compared with a response waveform of an electrical stimulus (pacing) to the heart, thereby identifying the position of the focus causing arrhythmia in the heart of the patient, and the doctor performs ablation on the position.

### CITATION LIST

### Patent Literature

[PTL 1]

Japanese Patent No. 5,160,245

### SUMMARY OF INVENTION

### Technical Problem

When the patient is to receive ablation treatment, the patient before the treatment tends to be in conditions of high tension. Therefore, there may occur a case where a signal waveform of arrhythmia cannot be acquired within a limited time period before the treatment. In such a case, the position of a focus for arrhythmia is hardly identified, and ablation treatment cannot be performed. To solve this problem, it may be contemplated that a long-term electrocardiogram is previously acquired outside a catheter inspection room by using, for example, a Holter electrocardiograph which can be carried by the patient, and a signal waveform of arrhythmia contained in the long-term electrocardiogram is used in ablation treatment.

Even when a signal waveform of arrhythmia contained in the long-term electrocardiogram which is acquired by using a Holter electrocardiograph or the like is compared with a response waveform of pacing, however, it is impossible to correctly identify the position of a focus for arrhythmia, and the long-term electrocardiogram cannot be used as it is in ablation treatment.

Present invention provides a cardiac catheter inspection apparatus, cardiac catheter inspection system, and cardiac catheter inspection method which can correctly identify the position of a focus for arrhythmia by using an electrocardiogram that is measured outside a catheter inspection room, and which enable ablation treatment to be performed.

### Solution to Problem

In order to achieve the above object, one aspect that the invention can take is a cardiac catheter inspection apparatus comprising: an acquiring section which is configured to, in a first electrocardiogram measured by a measurement apparatus that is placed outside a catheter inspection room, acquire a first waveform of heart beats during sinus rhythm, and an arrhythmia waveform during arrhythmia; a measuring section which is configured to measure a second electrocardiogram through electrodes attached to a patient who is waiting in the catheter inspection room; a first calculating section which is configured to calculate correction coefficients for statistically matching the first electrocardiogram and the second electrocardiogram with each other, based on the first waveform and a second waveform of heart beats during sinus rhythm in the second electrocardiogram; a waveform producing section which is configured to produce a comparison waveform that functions as an object to be compared with a response waveform of pacing performed by a cardiac catheter, based on the arrhythmia waveform contained in the first electrocardiogram, and the correction coefficients; and a second calculating section which is configured to calculate a matching rate of the response waveform and the comparison waveform.

And, another aspect that the invention can take is a cardiac catheter inspection system comprising: a measurement apparatus which is placed outside a catheter inspection room; an electrocardiogram analyzation apparatus which is configured to acquire and analyze a first electrocardiogram that is measured by the measurement apparatus, which is configured to extract a first waveform of heart beats during sinus rhythm, and an arrhythmia waveform during arrhythmia from the electrocardiogram, and which is configured to output the first waveform and the arrhythmia waveform to an outside; and the cardiac catheter inspection apparatus according to the above aspect.

And, another aspect that the invention can take is a cardiac catheter inspection method including the steps of: measuring a first electrocardiogram containing a first waveform of heart beats during sinus rhythm, and an arrhythmia waveform during arrhythmia, by a measurement apparatus that is placed outside a catheter inspection room; measuring a second electrocardiogram through electrodes attached to a patient who is waiting in the catheter inspection room; calculating correction coefficients for statistically matching the first electrocardiogram and the second electrocardiogram with each other, based on the first waveform and a second waveform of heart beats during sinus rhythm in the second electrocardiogram; producing a comparison waveform that functions as an object to be compared with a response waveform of pacing performed by a cardiac catheter, based on the arrhythmia waveform contained in the first electrocardiogram, and the correction coefficients; and calculating a matching rate of the response waveform and the comparison waveform.

According to the cardiac catheter inspection apparatus, cardiac catheter inspection system, and cardiac catheter inspection method of the invention, the position of a focus for arrhythmia can be correctly identified by using an electrocardiogram that is measured outside a catheter inspection room, and ablation treatment can be performed.

### BRIEF DESCRIPTION OF DRAWINGS

FIG. 1 is a diagram of a cardiac catheter inspection system of an embodiment of the invention.

FIG. 2 is a view illustrating a process of producing a second arrhythmia waveform.

FIG. 3 is a flowchart illustrating the operation of the cardiac catheter inspection system.

### DESCRIPTION OF EMBODIMENTS

Hereinafter, an embodiment of the invention will be described with reference to the drawings.

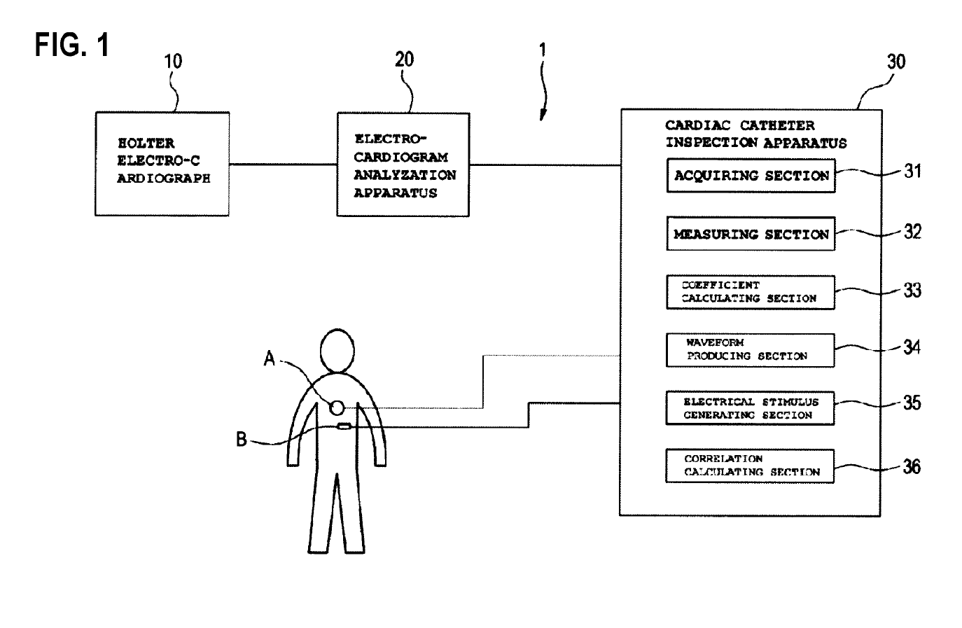

As shown in FIG. 1, a cardiac catheter inspection system 1 includes a Holter electrocardiograph (an example of the measurement apparatus) 10, an electrocardiogram analyzation apparatus 20, and a cardiac catheter inspection apparatus 30.

The Holter electrocardiograph 10 is an apparatus which can be carried by the patient, and which can measure an electrocardiogram during daily living. The electrocardiogram measures a first electrocardiogram which is, for example, a 24-hour electrocardiogram of the patient who has not yet received cardiac catheter treatment. The first electrocardiogram is an ML lead electrocardiogram. The ML lead (Mason-Likar lead) is a 12-lead by which an electrocardiogram corresponding to the standard 12-lead can be obtained by locating four-limb electrodes (R, L, F, RF) among the standard 12-lead electrodes in the body trunk. The lead is often employed in the case where an electrocardiogram is recorded while moving the four limbs. A bedside monitor which is to be placed at the side of the patient's bed may be used as the measurement apparatus. In this case, a standard 12-lead electrocardiogram is measured.

The electrocardiogram analyzation apparatus 20 analyzes the first electrocardiogram of the ML lead which is measured by the Holter electrocardiograph 10. The electrocardiogram analyzation apparatus 20 extracts, from the first electrocardiogram, a first sinus rhythm waveform (an example of the first waveform) which is the waveform of the heart beat during sinus rhythm, and a first arrhythmia waveform which is the waveform at the timing when arrhythmia occurs. The electrocardiogram analyzation apparatus 20 is communicably connected to the cardiac catheter inspection apparatus 30, and outputs the extracted first sinus rhythm waveform and first arrhythmia waveform, from an output terminal to the outside.

The cardiac catheter inspection apparatus 30 includes an acquiring section 31, a measuring section 32, a coefficient calculating section (an example of the first calculating section) 33, a waveform producing section 34, an electrical stimulus generating section 35, and a correlation calculating section (an example of the second calculating section) 36.

The acquiring section 31 acquires the first sinus rhythm waveforms of 12 leads and first arrhythmia waveforms of 12 leads which are output from the electrocardiogram analyzation apparatus 20. Moreover, the acquiring section 31 can produce a derived 18-lead electrocardiogram from the acquired first sinus rhythm waveforms of 12 leads.

The measuring section 32 measures a second electrocardiogram which is an electrocardiogram of the patient during cardiac catheter treatment. The second electrocardiogram is measured through biological electrodes A connected to the cardiac catheter inspection apparatus 30. In the embodiment, chest electrodes and four-limb electrodes for measuring a standard 12-lead electrocardiogram are used as the biological electrodes A. The biological electrodes A are attached to the body surface of the patient who is in a posture (supine posture) attained on a treatment table in a cardiac catheter inspection room.

The measuring section 32 extracts a second sinus rhythm waveform (an example of the second waveform) which is the waveform of the heart beat during sinus rhythm, from the second electrocardiogram. The measuring section 32 may produce a derived 18-lead electrocardiogram from the second sinus rhythm waveform. During the treatment, the measuring section 32 further extracts a response waveform (pseudo-waveform) which is generated by pacing performed by a cardiac catheter B. The cardiac catheter B is connected to the electrical stimulus generating section 35, and inserted into a blood vessel in, for example, the femoral region of the patient.

The coefficient calculating section 33 performs a matrix calculation by using the first sinus rhythm waveform and the second sinus rhythm waveform to calculate correction coefficients which cause the correlations of the waveforms to approximately coincide with each other. The correction coefficients are calculated respectively from the sinus rhythm waveforms of 12 leads which are measured by using, for example, six electrodes. The thus calculated twelve correction coefficients are used as the correction coefficients for statistically matching the first and second electrocardiograms with each other.

The waveform producing section 34 performs a matrix calculation on the first arrhythmia waveforms of 12 leads by using the correction coefficients which are calculated in the coefficient calculating section 33, to produce a second arrhythmia waveform which is an arrhythmia waveform that is presumed to be acquired from the patient who receives cardiac catheter treatment. In the embodiment, since a 12-lead electrocardiogram is used, the second arrhythmia waveform is calculated with respect to each of the first arrhythmia waveforms, and therefore twelve waveforms are produced in total. The second arrhythmia waveforms are used as comparison waveforms which are to be compared with a response waveform of pacing.

The electrical stimulus generating section 35 generates an electrical stimulus signal for performing pacing. The electrical stimulus signal which is output from the electrical stimulus generating section 35 is supplied to the cardiac catheter B. An electrical stimulus is applied to the myocardium from an electrode in the distal end of the cardiac catheter B which is inserted into the heart of the patient, whereby excitation of the myocardium is artificially induced, and a response waveform of pacing is generated in the electrocardiogram.

The correlation calculating section 36 calculates the matching rate of the response waveform of pacing and the second arrhythmia waveform. The matching rate is calculated for each of the waveforms of 12 leads.

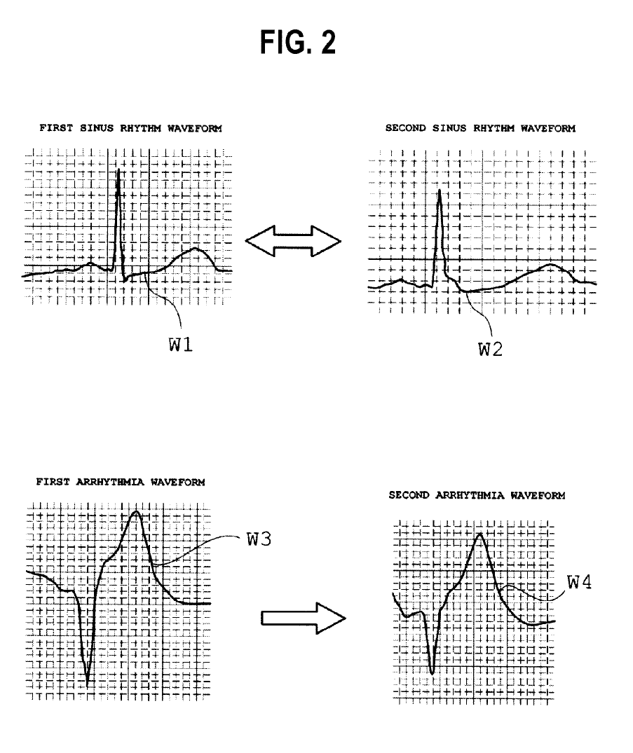

Next, a process of producing the second arrhythmia waveform will be described with reference to FIG. 2.

W1 in FIG. 2 shows an example of the first sinus rhythm waveform of the first electrocardiogram which is a waveform example of a part of 12 leads, and which is measured by the Holter electrocardiograph 10. W2 in FIG. 2 shows an example of the second sinus rhythm waveform of the second electrocardiogram which is a waveform example of a part of 12 leads, and which is measured through the biological electrodes A.

An electrocardiogram can be indicated by using electromotive force vectors centered at the heart. Therefore, the relationship between the first electrocardiogram and the second electrocardiogram can be indicated by following Math. 1.

$${V}_{i}^{’} = {\sum }_{j} {α}_{i , j} {V}_{j} \qquad [ Math . 1 ]$$

where V′ represents the waveform of the second electrocardiogram, V represents the waveform of the first electrocardiogram,

i represents each lead of 12 leads of the second electrocardiogram,

j represents each lead of 12 leads of the first electrocardiogram, and

{a_{i, j}} represents a matrix calculation.

The coefficient calculating section 33 substitutes the first sinus rhythm waveform and the second sinus rhythm in Math. 1 and performs a matrix calculation to calculate {a_{i, j}} that are the correction coefficients which cause the correlations of the waveforms to approximately coincide with each other.

W3 in FIG. 2 shows an example of the first arrhythmia waveforms of the first electrocardiogram which is a waveform example of a part of 12 leads, and which is measured by the Holter electrocardiograph 10.

The waveform producing section 34 performs a matrix calculation on the first arrhythmia waveforms by using {a_{i, j}} which are the correction coefficients, to obtain an arrhythmia waveform W4 such as shown in FIG. 2. The thus obtained arrhythmia waveform is a second arrhythmia waveform W4 which is presumed to be measured through the biological electrodes A.

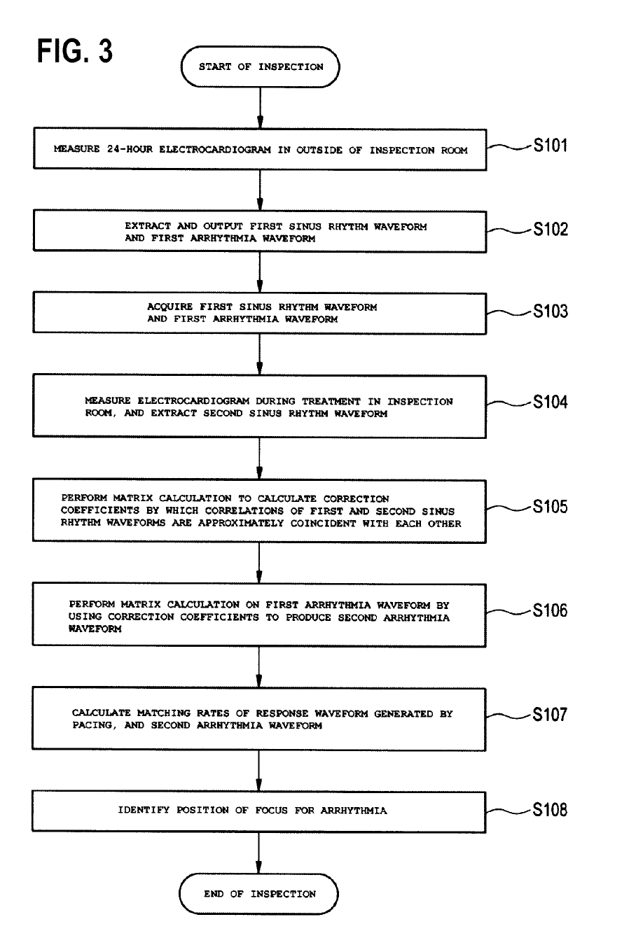

Next, a cardiac catheter inspection method using the cardiac catheter inspection system 1 will be described with reference to FIG. 3.

In a state where the patient before treatment tends is not in conditions of high tension, i.e., the patient is in usual living conditions, firstly, the first electrocardiogram containing the first sinus rhythm waveform and first arrhythmia waveform of the patient is previously measured by the Holter electrocardiograph 10 which is carried by the patient outside the cardiac catheter inspection room (step S101).

After the measurement, the electrocardiogram analyzation apparatus 20 reads the first electrocardiogram measured by the Holter electrocardiograph 10, extracts the first sinus rhythm waveform and the first sinus rhythm waveform from the first electrocardiogram, and outputs the extracted waveforms to the cardiac catheter inspection apparatus 30 (step S102).

The first sinus rhythm waveform and first arrhythmia waveform which are output from the electrocardiogram analyzation apparatus 20 are acquired by the acquiring section 31 of the cardiac catheter inspection apparatus 30 (step S103).

Next, the second electrocardiogram is measured by the measuring section 32 from the patient who is waiting for cardiac catheter treatment in a supine posture on the treatment table in the inspection room, and the second sinus rhythm waveform is extracted from the second electrocardiogram (step S104).

Then, a matrix calculation is performed by using the first sinus rhythm waveforms of 12 leads acquired in step S103, and the second sinus rhythm waveforms of 12 leads extracted in step S104, and, for each lead, the correction coefficient for causing the correlation of the first and second sinus rhythm waveforms to approximately coincide with each other is calculated by the coefficient calculating section 33 (step S105).

Then, a matrix calculation is performed on the first arrhythmia waveforms of 12 leads acquired in step S103 by using the correction coefficients calculated in step S105, and the second arrhythmia waveforms (comparison waveforms) corresponding respectively to the first arrhythmia waveforms are produced by the the waveform producing section 34 (step S106).

Then, pacing is performed by using the cardiac catheter B inserted into the heart of the patient. The myocardial excitation induced by the pacing is measured as the response waveform contained in the second electrocardiogram, through the biological electrodes A by the measuring section 32, and extracted from the second electrocardiogram by the measuring section 32. The matching rates of the extracted response waveforms of 12 leads and the second arrhythmia waveforms of 12 leads which are produced in step S106 are calculated by the correlation calculating section 36 (step S107).

The response waveforms generated by the pacing, the matching rates of the response waveforms and the second arrhythmia waveforms, and the like are displayed on a displaying section (not shown) disposed in the cardiac catheter inspection apparatus 30. While checking the response waveforms, matching rates, and the like which are displayed on the displaying section, the pacing by the cardiac catheter B is repeatedly performed. Based on the matching rates of the standard electrocardiograms of 12 leads which are calculated in the pacing modes, the response waveform of the highest matching rate is comprehensively specified. Then, the position where the specified response waveform is generated is identified as the origin (focus) of generation of arrhythmia (step S108). Ablation treatment is performed on the portion of the identified focus.

The patient before ablation treatment tends to be in conditions of high tension. Therefore, there may occur a case where, because of the influence of the above, a signal waveform of arrhythmia which is spontaneously generated from the patient cannot be acquired within a limited time period before the treatment. In the case where there are a plurality of focuses for arrhythmia, it is necessary to acquire signal waveforms of arrhythmia which are spontaneously generated based on the respective focuses. However, there is a case where it is difficult to acquire signal waveforms of arrhythmia which are spontaneously generated based on all the focuses, within a limited time period before the treatment. In the case where it is impossible to acquire an electrocardiogram waveform which functions as the object of the comparison with a response waveform of pacing using a cardiac catheter, it is difficult to correctly identify the position of a focus for arrhythmia, and ablation treatment cannot be performed.

Therefore, it may be contemplated that a long-term electrocardiogram is previously acquired outside a catheter inspection room by using, for example, a Holter electrocardiograph, and a signal waveform of arrhythmia contained in the long-term electrocardiogram is used in ablation treatment. Between the electrocardiogram measurement using a Holter electrocardiograph, and that performed in a catheter inspection room, however, the positions where electrodes for acquiring an electrocardiogram are applied, and the posture of the patient when an electrocardiogram is measured are different, and therefore the shapes of the waveforms of the electrocardiograms acquired in the measurements are not always identical with each other. Even when the arrhythmia waveform contained in the long-term electrocardiogram acquired by using a Holter electrocardiograph is compared with the response waveform of pacing, therefore, the position of a focus for arrhythmia cannot be correctly identified. In the conventional art, as described above, the long-term electrocardiogram cannot be used as it is in ablation treatment.

In the embodiment, according to the cardiac catheter inspection system 1, the cardiac catheter inspection apparatus 30, and the cardiac catheter inspection method, by contrast, the second arrhythmia waveform which is presumed to be generated during the catheter inspection is produced for each lead by a calculation process by using the first sinus rhythm waveform and first arrhythmia waveform contained in the first electrocardiogram of 24 hours which is previously measured outside the catheter inspection room.

Even when the patient who is waiting in the catheter inspection room is in conditions of high tension and therefore the waveform of spontaneous arrhythmia cannot be acquired, the heartbeat waveform (second sinus rhythm waveform) of the patient during the sinus rhythm can be acquired. From the second sinus rhythm waveform and the first sinus rhythm waveform which is previously measured, therefore, the correction coefficients by which the correlations of the waveforms are approximately coincident with each other are calculated. Then, a calculation is performed on the first arrhythmia waveform which is previously measured, by using the correction coefficients, and the second arrhythmia waveform which is presumed to be generated during the catheter inspection is produced. Even when conditions for measuring the first and second electrocardiograms, such as the positions where electrodes of 12 leads for acquiring an electrocardiogram are applied, and the posture of the patient when an electrocardiogram is measured are different from each other, therefore, the second arrhythmia waveform which is highly correlated with the arrhythmia waveform of the patient that is spontaneously generated during the catheter inspection can be produced without being affected by the differences. When pacing is performed by using the thus produced second arrhythmia waveform, the position where ablation treatment is to be performed can be identified accurately and correctly.

In the embodiment, the correction coefficient is obtained with respect to the waveform in each lead of a 12-lead electrocardiogram, and the second arrhythmia waveform is produced for each lead. Therefore, the position where ablation treatment is to be performed can be accurately identified.

When the Holter electrocardiograph 10 which is configured as a portable apparatus is used, it is possible to acquire an electrocardiogram of a long term including active and inactive (such as sleeping) times. Alternatively, a bedside monitor may be used. In the alternative, for example, a long-term electrocardiogram of a hospitalized patient can be previously acquired. Therefore, an arrhythmia waveform of the patient can be acquired previously and surely. Even in the case where there are a plurality of focuses for arrhythmia, when a long-term electrocardiogram is used, arrhythmia waveforms which are spontaneously generated based on the respective focuses can be previously acquired without omission.

Even in the case where the patient who is waiting in the catheter inspection room is in conditions of high tension and therefore an arrhythmia waveform cannot be acquired, when the matching rate of the produced second arrhythmia waveform and the response waveform of pacing is checked as described above, the doctor can correctly identify the position of the focus for arrhythmia in cardiac catheter treatment, and surely treat the focus by ablation.

The invention is not limited to the above-described embodiment, and may be adequately subjected to modifications, improvements, and the like. In addition, the materials, shapes, dimensions, values, forms, numbers, places, and the like of the components of the above-described embodiment are arbitrary and not limited insofar as the invention is achieved.

Although, in the above, the embodiment in which an electrocardiogram containing a plurality of heartbeat waveforms of 12 leads or 18 leads is used has been described, for example, the invention is not limited to the embodiment. The invention can be applied to any electrocardiogram as far as it contains at least one heartbeat waveform.

In the cardiac catheter inspection system 1 of the embodiment, the manner of allocating the functions of the system to the apparatuses can be arbitrarily selected. For example, the system may be configured so that the cardiac catheter inspection apparatus 30 has the function of extracting the first sinus rhythm waveform and the first arrhythmia waveform from the first electrocardiogram.

The present application is based on Japanese Patent Application No. 2015-003225 filed on Jan. 9, 2015, the contents of which are hereby incorporated by reference.

1. A cardiac catheter inspection apparatus comprising:
an acquiring section which is configured to, in a first electrocardiogram measured by a measurement apparatus that is placed outside a catheter inspection room, acquire a first waveform of heart beats during sinus rhythm, and an arrhythmia waveform during arrhythmia;
a measuring section which is configured to measure a second electrocardiogram through electrodes attached to a patient who is waiting in the catheter inspection room;
a first calculating section which is configured to calculate correction coefficients for statistically matching the first electrocardiogram and the second electrocardiogram with each other, based on the first waveform and a second waveform of heart beats during sinus rhythm in the second electrocardiogram;
a waveform producing section which is configured to produce a comparison waveform that functions as an object to be compared with a response waveform of pacing performed by a cardiac catheter, based on the arrhythmia waveform contained in the first electrocardiogram, and the correction coefficients; and
a second calculating section which is configured to calculate a matching rate of the response waveform and the comparison waveform.

2. The cardiac catheter inspection apparatus according to claim 1, wherein the first electrocardiogram and the second electrocardiogram are 12-lead electrocardiograms.

3. A cardiac catheter inspection system comprising:
a measurement apparatus which is placed outside a catheter inspection room;
an electrocardiogram analyzation apparatus which is configured to acquire and analyze a first electrocardiogram that is measured by the measurement apparatus, which is configured to extract a first waveform of heart beats during sinus rhythm, and an arrhythmia waveform during arrhythmia from the electrocardiogram, and which is configured to output the first waveform and the arrhythmia waveform to an outside; and
a cardiac catheter inspection apparatus including:
an acquiring section which is configured to, in the first electrocardiogram, acquire the first waveform, and the arrhythmia waveform;
a measuring section which is configured to measure a second electrocardiogram through electrodes attached to a patient who is waiting in the catheter inspection room;
a first calculating section which is configured to calculate correction coefficients for statistically matching the first electrocardiogram and the second electrocardiogram with each other, based on the first waveform and a second waveform of heart beats during sinus rhythm in the second electrocardiogram;
a waveform producing section which is configured to produce a comparison waveform that functions as an object to be compared with a response waveform of pacing performed by a cardiac catheter, based on the arrhythmia waveform contained in the first electrocardiogram, and the correction coefficients; and
a second calculating section which is configured to calculate a matching rate of the response waveform and the comparison waveform.

4. The cardiac catheter inspection system according to claim 3, wherein the measurement apparatus is a portable Holter electrocardiograph.

5. The cardiac catheter inspection system according to claim 3, wherein the measurement apparatus is a bedside monitor.

6. A cardiac catheter inspection method including the steps of:
measuring a first electrocardiogram containing a first waveform of heart beats during sinus rhythm, and an arrhythmia waveform during arrhythmia, by a measurement apparatus that is placed outside a catheter inspection room;
measuring a second electrocardiogram through electrodes attached to a patient who is waiting in the catheter inspection room;
calculating correction coefficients for statistically matching the first electrocardiogram and the second electrocardiogram with each other, based on the first waveform and a second waveform of heart beats during sinus rhythm in the second electrocardiogram;
producing a comparison waveform that functions as an object to be compared with a response waveform of pacing performed by a cardiac catheter, based on the arrhythmia waveform contained in the first electrocardiogram, and the correction coefficients; and
calculating a matching rate of the response waveform and the comparison waveform.

7. The cardiac catheter inspection system according to claim 3, wherein the first electrocardiogram and the second electrocardiogram are 12-lead electrocardiograms.
